(19) United States
(12) Patent Application Publication
(10) Pub. No.: US 2006/0258589 A1
(43) Pub. Date: Nov. 16, 2006

(54) Methods for enhancing radiation therapy

(75) Inventor: Hugh McTavish, Birchwood, MN (US)

Correspondence Address: Hugh McTavish;McTavish Patent Firm, 429 Birchwood Courts, Birchwood, MN, 55110, US

(73) Assignee: IGF Oncology, LLC, Birchwood, MN (US)

(21) Appl. No.: 11/490,623

(22) Filed: Jul. 21, 2006

Related U.S. Application Data
(63) Continuation-in-part of application No. PCT/US05/02110, filed on Jan. 24, 2005.
(60) Provisional application No. 60/539,203, filed on Jan. 24, 2004.

(51) Int. Cl. A61K 38/30

(52) U.S. Cl. 514/12

(57) ABSTRACT

Methods of treating cancer are provided. The methods are improved methods of radiation therapy involving administering an IGF-1-receptor agonist along with therapeutic radiation to a mammal afflicted with cancer. The IGF-1-receptor agonist causes the cancer cells to divide and move into more sensitive stages of the cell cycle, sensitizing the cancer cells to be killed more efficiently by radiation. Also provided are methods of treating cancer involving administering radiation to a mammal together with administering a growth-hormone agonist, an insulin-receptor agonist, or sugar.

This application is a continuation-in-part application of international application no. PCT/US2005/002110, filed Jan. 24, 2005, and claims priority to U.S. provisional patent application Ser. No. 60/539,203, filed Jan. 24, 2004.

### BACKGROUND

Radiation therapy against cancer has at least two key limitations to its effectiveness. The first is the side effects from killing non-target (i.e., non-cancerous) cells. Radiation therapy kills cancer cells by damaging key molecules in the cell, particularly DNA. Radiation can cause damage directly (e.g., by ionizing one of the atoms of the DNA molecule and this leading to strand breakage) or indirectly (e.g., by ionizing water and causing a chain of events that leads to free radical formation, where the free radicals then damage DNA or other cellular components). By either direct or indirect mechanisms, radiation is more toxic to dividing cells than non-dividing cells. Cancer is characterized by cells that divide inappropriately and in an uncontrolled manner. However, radiation kills all dividing cells, whether cancerous or not. This accounts for the side-effects of radiation, including immune suppression when bone marrow or other immune progenitor cells are irradiated, and nausea, when the gastrointestinal tract is irradiated. With a high enough dose of radiation, it is always possible to kill a tumor. The trick is to do it without killing the patient—that is, without causing an unacceptable level of damage to surrounding non-cancerous cells. Thus, the side-effects are more than just an unpleasant experience for the patient—they limit the dose and effectiveness of treatment.

The second limitation of radiation's effectiveness is that non-dividing cells and cells in certain stages of the cell cycle are markedly less sensitive to radiation. Dividing cells are more sensitive to radiation at least in part because radiation is more lethal to cells in the G2 and M phases of the cell cycle, and these phases are associated with dividing cells. Cells in other phases of the cell cycle are less sensitive. Cancer cells inappropriately divide, but they are not constantly dividing. Thus, any time radiation is administered some fraction of the cancer cells will not be dividing and will be comparatively insensitive to radiation damage.

Methods to enhance the effectiveness of radiation treatment for cancer are needed. Preferably the methods would decrease the side effects of treatment. Preferably the methods would enhance the lethality of radiation treatment for cancerous cells, while causing less or no increase in the lethality of the radiation to non-cancerous cells.

### SUMMARY

In one embodiment of the invention, insulin-like growth factor-1 (IGF-1) (or an IGF-1-receptor agonist or growth hormone, which stimulates IGF-1 release) is administered to a patient before, during, or after treating the patient with radiation. Preferably, the IGF-1-receptor agonist or growth hormone is administered shortly before or immediately before treating the patient with radiation. Preferably, the radiation is externally applied, as opposed to an implanted radioisotope, so that the time span between binding IGF-1 to the cells and irradiating the cells can be controlled.

Cancer cells of most or nearly all types of cancers have more IGF-1 receptors than normal cells of the same tissue type. Upon binding to IGF-1 receptors, IGF-1 stimulates cells to divide. Thus, by causing cells to divide, IGF-1 enhances the sensitivity of the cells to radiation. Since cancer cells have more IGF-1 receptors than non-cancer cells, this effect will be greater on cancer cells. Thus, administering IGF-1 increases the sensitivity of cancer cells more than non-cancer cells to radiation and increases the selectivity of radiation therapy.

IGF-1 or another IGF-1-receptor agonist can be administered directly to the patient. Alternatively, growth hormone, which stimulates IGF-1 production and release in the body, can be administered.

This method is effective against any type of cancer where the cancer cells have IGF-1 receptors and are responsive to IGF-1 binding. Preferably, the cancer cells have an elevated number of IGF-1 receptors (i.e., more receptors than normal cells of the same tissue type).

IGF-1, another IGF-1-receptor agonist, or growth hormone can be administered systemically or locally. For instance, for local administration the agonist could be injected directly into a tumor.

In addition to stimulating cancer cells to divide, IGF-1 moves a greater proportion of the cells into the G2 and M phases of the cell cycle. (Ciftci, K., 2003, J. Pharmacy Pharmacol. 55:1135.) These are the phases of the cell cycle when cells are most sensitive to radiation. (Waldow, Stephen M., Overview of Radiobiology, Chapter 9 in Introduction to Radiation Therapy.)

IGF-1, by stimulating cancers to divide, makes them more aggressive and can promote metastasis. Thus, it would be unwise to administer IGF-1 in the absence of treatment. But if IGF-1 is administered only in conjunction with radiation therapy (or chemotherapy) it will promote killing of the cancer cells by the radiation (or chemotherapy).

In conjunction with administering IGF-1 at or near the time of radiation treatment, an IGF-1-receptor antagonist can be administered between treatment sessions in order to inhibit the cancer cells from dividing or metastasizing between treatment sessions.

To reduce the danger of some of the cancer cells stimulated by IGF-1 surviving the radiation treatment and then becoming more aggressive, instead of administering IGF-1 (or an IGF-1-receptor agonist or growth hormone) to the patient, an IGF-1-chemotherapeutic agent conjugate (e.g., IGF-1-methotrexate) can be administered to the patient in conjunction with radiation treatment. See U.S. provisional patent application 60/513,048, filed Oct. 21, 2003, and U.S. utility patent application Ser. No. 11/407,590, filed Apr. 20, 2006, for a description of the conjugates. If a conjugate is administered instead of IGF-1, any cells stimulated by the IGF-1 portion of the conjugate will take up the chemotherapeutic agent along with IGF-1. Thus, they are less likely to survive the treatment. (The conjugate could also be a conjugate of another IGF-1-receptor agonist, instead of IGF-1 itself, to a chemotherapeutic agent.) Insulin has properties similar to IGF-1. For one thing, insulin and IGF-1 are homologous (evolutionarily related) proteins. They cross-react to each other's receptors. Insulin has been shown to enhance the killing of breast cancer cells in tissue culture by up to 10,000 fold. This does not appear to be because insulin enhances uptake of methotrexate. Another study found it only enhanced uptake of methotrexate by a factor of 2. Thus, the more likely hypothesis is that insulin enhances methotrexate killing by stimulating the cancer cells to divide, and thus making them more sensitive. Insulin is also known to stimulate cells bearing insulin receptors to divide. In part, insulin's effect of stimulating cells to divide may be because insulin binds to IGF-1 receptors (although at a lower affinity than IGF-1 does). Cancer cell of most or nearly all types of cancer have more insulin receptors, as well as IGF-1 receptors, than normal cells of the same tissue type.

Thus, the invention also provides for administering insulin or another insulin-receptor agonist to a patient immediately before, or shortly before, radiation treatment, instead of or together with administering IGF-1. In addition, just as IGF-1 release could be stimulated by administering growth hormone, insulin release could be stimulated by administering sugar, either orally or intravenously, to the patient. Thus, the invention provides for administering sugar to a patient immediately before or shortly before radiation treatment. The combination of insulin and sugar can also be more effective than either alone in stimulating activity of cancer cells and sensitizing the cancer cells to radiation therapy.

Using insulin to enhance radiation effectiveness is effective against any type of cancer where the cancer cells have insulin receptors and are responsive to insulin. Preferably the cancer cells have an elevated number of insulin receptors (i.e., more receptors than normal cells of the same tissue type).

Insulin or an insulin-receptor agonist can be administered systemically or locally. For instance, for local administration insulin or the agonist could be injected directly into a tumor.

Insulin, by stimulating cancer cells to divide, can make them more aggressive and can promote metastasis. Thus, it would be unwise to administer insulin in the absence of radiation treatment or chemotherapy. Between treatments, in order to prevent stimulating the cancer cells to divide and be active, the patient should be advised to minimize sugar consumption (and thus insulin production). Also, an insulin-receptor antagonist could be administered.

To reduce the danger of some of the cancer cells stimulated by insulin surviving the radiation treatment and then becoming more aggressive, instead of administering insulin (or an insulin-receptor agonist or sugar) to the patient, an insulin-chemotherapeutic agent conjugate (e.g., insulin-methotrexate) can be administered to the patient in conjunction with radiation treatment. See U.S. provisional patent application 60/513,048, filed Oct. 21, 2003, for a description of the conjugates. If a conjugate is administered instead of insulin, any cells stimulated by the insulin portion of the conjugate will take up the chemotherapeutic agent along with insulin. Thus, they are less likely to survive the treatment. (The conjugate could also be a conjugate of an insulin-receptor agonist, instead of insulin itself, to a chemotherapeutic agent.) One aspect of the invention is enhancing the effectiveness of radiation therapy by coadministering both (1) IGF-1, another IGF-1-receptor agonist, or growth hormone; and (2) insulin, another insulin-receptor agonist, or sugar, before during or after administering radiation to a mammal afflicted with cancer. While most cancer cells have an elevated number of IGF-1 receptors and most have an elevated number of insulin receptors, some may be more elevated in one than the other, and some may be more responsive to one than the other. Thus, it can be advantageous to administer both an insulin-receptor agonist and an IGF-1-receptor agonist. Administering both is insurance against the cancer cells being more responsive to one than the other. Furthermore, the effects of an insulin-receptor agonist and an IGF-1-receptor agonist in enhancing the effectiveness of radiation are expected to be additive.

Thus, the invention provides a method of treating cancer in a mammal involving: administering an agent containing an IGF-1-receptor agonist to the mammal and administering radiation to the mammal.

Another embodiment of the invention provides a method of enhancing the effectiveness of anti-cancer radiation therapy in a mammal involving: administering an agent containing an IGF-1-receptor agonist to the mammal before, during, or after administering radiation to the mammal.

Another embodiment of the invention provides a method of screening for agents that enhance the effectiveness of radiation therapy, the method involving: (a) contacting cancer cells with an IGF-1-receptor agonist-chemotherapeutic agent conjugate and irradiating the cancer cells, and measuring the survival of the cancer cells; (b) irradiating the cancer cells wherein the cancer cells are not contacted with the conjugate, and measuring the survival of the cancer cells; and (c) comparing the survival of the cancer cells in (a) and (b).

Another embodiment of the invention provides a method of screening for agents that enhance the effectiveness of radiation therapy, the method involving: (a) contacting cancer cells with an insulin-receptor agonist-chemotherapeutic agent conjugate and irradiating the cancer cells, and measuring the survival of the cancer cells; (b) irradiating the cancer cells wherein the cancer cells are not contacted with the conjugate, and measuring the survival of the cancer cells; and (c) comparing the survival of the cancer cells in (a) and (b).

Another embodiment of the invention provides a method of treating cancer in a mammal involving: administering a growth hormone-receptor agonist to the mammal and administering radiation to the mammal.

Another embodiment of the invention provides a method of enhancing the effectiveness of anti-cancer radiation therapy in a mammal comprising: administering a growth hormone-receptor agonist to the mammal before, during, or after administering radiation to the mammal.

Another embodiment of the invention provides a method of treating cancer in a mammal involving: administering an insulin-receptor agonist-chemotherapeutic agent conjugate to the mammal before, during, or after administering radiation to the mammal.

Another embodiment of the invention provides a method of enhancing the effectiveness of radiation therapy in a mammal involving: administering an insulin-receptor agonist-chemotherapeutic agent conjugate to the mammal before, during, or after administering radiation to the mammal.

Another embodiment of the invention provides a method of treating cancer in a mammal involving: administering an agent containing an IGF-1-receptor agonist or administering growth hormone to the mammal; administering an agent containing an insulin-receptor agonist or administering sugar to the mammal; and administering radiation to the mammal.

Another embodiment of the invention provides a method of enhancing the effectiveness of anti-cancer radiation therapy in a mammal involving: administering an agent comprising an IGF-1-receptor agonist or growth hormone to the mammal before, during, or after administering radiation to the mammal; and administering an agent comprising an insulin-receptor agonist or sugar to the mammal before, during, or after administering radiation to the mammal.

### DETAILED DESCRIPTION

### Definitions

The terms “chemotherapeutic agent” or “anti-cancer chemotherapeutic agent” are used interchangeably herein. The terms refer to a synthetic, biological, or semi-synthetic compound that kills cancer cells or inhibits the growth of cancer cells while having less effect on non-cancerous cells. The terms include enzymes that have anti-cancer properties, e.g., asparaginase, and photoactivatable anti-cancer agents, e.g., chlorin e-6.

The term “treating cancer” includes, e.g., preventing metastasis, inhibiting growth of a cancer, or stopping the growth of cancer, as well as killing a tumor.

The term “binding affinity” of a ligand for a particular receptor refers to the association constant K_{A} (the inverse of the dissociation constant K_{D}) or to experimentally determined approximations thereof.

The term “agonist” refers to a ligand to the insulin receptor or IGF-1 receptor that, when it binds to the receptor, activates the normal biochemical and physiological events triggered by binding of the natural ligand for the receptor (i.e, insulin for the insulin receptor or IGF-1 for the IGF-1 receptor). In particular embodiments, an agonist has at least 20%, at least 30%, or at least 50% of the biological activity of the natural ligand. The activity of an insulin receptor ligand can be measured, for instance, by measuring the hypoglycemic effect (Poznansky, M. J., et al., 1984, Science 223:1304). The activity of an insulin-receptor ligand or IGF-1-receptor ligand can be measured in vitro by the measuring the extent of autophosphorylation of the receptor in response to ligand binding, as described in Satyamarthy, K., et al., 2001, Cancer Res. 61:7318. MAP kinase phosphorylation can also be measured for the IGF-1 receptor (Satyamarthy, K., et al., 2001, Cancer Res. 61:7318).

The term “antagonist” refers to a ligand that has little or no stimulating activity when it binds to the receptor and that inhibits or prevents binding of the natural ligand to the receptor. In particular embodiments, an antagonist has less than 20%, less than 10%, or less than 5% of the activity of the natural ligand (insulin for the insulin receptor or IGF-1 for the IGF-1 receptor).

“Containing” as used herein is open-ended; i.e., it allows the inclusion of other unnamed elements and has the same meaning as “comprising.”

### Description

The invention involves administering to a mammal afflicted with cancer radiation and one or more agents that sensitize cancer cells in the mammal to killing by the radiation. The sensitizing agents can be an IGF-1-receptor agonist, an insulin-receptor agonist, growth hormone (which causes the release of IGF-1 in the mammal), and/or a sugar (which causes the release of insulin in the mammal).

The agents can be administered before, during, or after administration of the radiation. The agents are administered close enough in time to the radiation to enhance the effectiveness of the radiation in killing cancer cells. This is typically at least within 12 hours before or after administration of the radiation. Preferably, the agents are administered 0 to 6 hours before the radiation is administered. More preferably, the agents are administered 0 to 3 hours or 15 minutes to 3 hours before the radiation is administered. In particular embodiments, the agents are administered between 6 hours before and 6 hours after the radiation is administered. In another particular embodiment, the agents are administered between 3 hours before and 3 hours after the radiation is administered.

One embodiment of the invention provides a method of treating cancer in a mammal comprising: administering an agent comprising an IGF-1-receptor agonist to the mammal and administering radiation to the mammal.

In particular embodiments, the IGF-1-receptor agonist is IGF-1. In another particular embodiment, the agent consists of IGF-1.

In particular embodiments, the IGF-1-receptor agonist is not an insulin-receptor agonist.

In specific embodiments, the IGF-1-receptor agonist has a K_{D} for the insulin receptor of greater than 0.5 nM, greater than 1 nM, or greater than 2 nM.

In specific embodiments, the IGF-1-receptor agonist has a binding affinity for the IGF-1 receptor greater than insulin. In specific embodiments, the IGF-1-receptor agonist has a binding affinity for the IGF-1 receptor greater than for the insulin receptor.

In particular embodiments of the invention, the IGF-1-receptor agonist or antagonist has a K_{D} for the IGF-1 receptor of less than 1 mM, less than 100 μM, less than 10 μM, less than 1 μM, less than 100 nM, less than 50 nM, less than 20 nM, less than 10 nM, less than 5 nM, less than 2 nM, or less than 1 nM.

In particular embodiments, the IGF-1-receptor agonist is not IGF-1.

The IGF-1-receptor agonist can be a peptide in some embodiments. For instance, it can be a peptide of 2-60 amino acid residues, of 2-40 amino acid residues, of 2-20 amino acid residues, of 5-60 amino acid residues, of 5-40 amino acid residues, or of 5-20 amino acid residues.

In particular embodiments of the methods using an agent that comprises an IGF-1-receptor agonist or an insulin-receptor agonist, the agent is, or comprises, an IGF-1-receptor agonist-chemotherapeutic agent conjugate or an insulin-receptor agonist-chemotherapeutic agent conjugate.

The chemotherapeutic agent portion of the conjugates in particular embodiments is amsacrine, azacytidine, bleomycin, busulfan, capecitabine, carboplatin, carmustine, chlorambucil, cisplatin, cladribine, cyclophosphamide, cytarabine, dactinomycin, daunorubicin, decarbazine, docetaxel, doxorubicin, epirubicin, estramustine, etoposide, floxuridine, fludarabine, fluorouracil, gemcitabine, hexamethylmelamine, idarubicin, ifosfamide, irinotecan, lomustine, mechlorethamine, melphalan, mercaptopurine, mitomycin C, mitotane, mitoxantrone, oxaliplatin, paclitaxel, pemetrexed, pentostatin, plicamycin, procarbazine, ralitrexed, semustine, streptozocin, temozolamide, teniposide, thioguanine, thiotepa, topotecan, trimitrexate, valrubicin, vincristine, vinblastine, vindestine, or vinorelbine.

Other examples of IGF-1-receptor agonists include variants of IGF-1 that activate the receptor but have reduced affinity for the soluble IGF-1 binding proteins, such as those disclosed in U.S. Pat. No. 4,876,242. IGF binding proteins are natural serum proteins that bind to IGF-1, holding it in circulation and extending its biological half-life. It may be advantageous for the IGF-1 receptor ligands of this invention to have reduced binding to the IGF-1 binding proteins, because that reduced binding would accelerate the release of the agent to bind to the IGF-1 receptors. Thus, in some embodiments, the IGF-1 receptor ligand or agonist has reduced affinity for soluble IGF-1 binding proteins, as compared to native IGF-1.

One preferred variant IGF-1 for use in the methods and conjugates of the invention that has reduced binding affinity for the soluble IGF-1 binding proteins is LONG-R3-IGF-1 (Francis, G. L., et al.1992, J Mol. Endocrinol. 8:213-223; Tomas, F. M. et al., 1993, .J. Endocrinol. 137:413-421) (SEQ ID NO:1). SEQ ID NO:1 has the sequence MFPAMPLSSL FVNGPRTLCG AELVDALQFV CGDRGFYFNK PTGYSSSRRA PQTGIVDECC FRSCDLRRLE MYCAPLKPAK SA.

Preferably, the IGF-1 receptor ligand with reduced affinity for soluble IGF-1 binding proteins has at least 5-fold, more preferably at least 10-fold, more preferably still at least 100-fold lower binding affinity for soluble IGF-1 binding proteins than wild-type IGF-1. Binding affinity for the soluble IGF-1 binding proteins can be measured by a competition binding assay against labeled IGF-1 (e.g., I-125-IGF-1), using a mixture of purified IGF-1 binding proteins or rat L6 myoblast-conditioned medium (a naturally produced mixture of IGF-1 binding proteins), as described in Francis, G. L., et al. (1992, J Mol. Endocrinol. 8:213-223); Szabo, L. et al. (1988, Biochem. Biophys. Res. Commun. 151:207-214); and Martin, J. L. et al. (1986, J Biol. Chem. 261:8754-8760). Preferably, the variant IGF-1 has an IC₅₀ in a competition binding assay against labeled wild-type IGF-1 for binding to soluble IGF-1 binding proteins in L6 myoblast-conditioned medium of greater than 10 nM, more preferably greater than 100 nM.

Preferably, the variant IGF-1 with reduced affinity for soluble IGF-1 binding proteins has affinity for the IGF-1 receptor that is close to wild-type IGF-1 (e.g., less than 30-fold greater than wild-type IGF-1, more preferably less than 10-fold greater than wild-type IGF-1). In specific embodiments, the variant IGF-1 has an IC₅₀ in a competition binding assay against labeled wild-type IGF-1 for binding to IGF-1 receptors (e.g., on MCF-7 cells) of less than 50 nM, more preferably less than 10 nM, more preferably still less than 5 nM, more preferably still less than 3 nM). This assay is described in Ross, M. et al. (1989, Biochem. J. 258:267-272) and Francis, G. L., et al. (1992, J. Mol. Endocrinol. 8:213-223).

In embodiments of the invention involving use of an agent comprising an insulin-receptor agonist, the insulin-receptor agonist may be insulin.

In particular embodiments, the insulin-receptor agonist is glycyl-L-histidyl-L-lysine-acetate (Biaglow, J. E., et al., 1979, Int. J. Radiat. Oncol. Biol. Phys. 5:1669).

In particular embodiments of the invention, the insulin-receptor agonist is not an IGF-1-receptor agonist.

In specific embodiments, the insulin-receptor agonist has a K_{D} for the IGF-1 receptor of greater than 0.5 nM, greater than 1 nM, or greater than 2 nM.

In specific embodiments, the insulin-receptor agonist has a binding affinity for the insulin receptor greater than IGF-1.

In particular embodiments of the invention, the insulin-receptor agonist or antagonist has a K_{D} for the insulin receptor of less than 1 mM, less than 100 μM, less than 10 μM, less than 1 μM, less than 100 nM, less than 50 nM, less than 20 nM, less than 10 nM, less than 5 nM, less than 2 nM, or less than 1 nM.

The insulin-receptor agonist can be a peptide in some embodiments. For instance, it can be a peptide of 2-60 amino acid residues, of 2-40 amino acid residues, of 2-20 amino acid residues, of 5-60 amino acid residues, of 5-40 amino acid residues, or of 5-20 amino acid residues.

In particular embodiments of the invention, the methods are used to treat lung cancer (small cell or non-small cell), prostate cancer, colorectal cancer, breast cancer, pancreatic cancer, leukemia, liver cancer, stomach cancer, ovarian cancer, uterine cancer, testicular cancer, brain cancer, non-Hodgkin's lymphoma, Hodgkin's lymphoma, Ewing's sarcoma, osteosarcoma, neuroblastoma, rhabdomyosarcoma, melanoma, head or neck cancer, or brain cancer.

The methods of the invention may be particularly suited for treatment of low-grade non-Hodgkin's lymphoma. Low-grade non-Hodgkin's lymphoma is less curable than intermediate-grade or aggressive-grade non-Hodgkin's lymphoma because the cancer cells in low-grade non-Hodgkin's lymphoma divide less frequently and are thus less susceptible to radiation therapy than intermediate and aggressive-grade lymphomas. The agents described herein will cause the tumor cells to divide more frequently and thus be more sensitive to radiation killing.

In particular embodiments of the methods described herein, the mammal treated by the methods is a human. In other embodiments, the mammal is an experimental mammal, e.g., a mouse. In particular embodiments, the mammal is a dog, cat, rabbit, guinea pig, or pig.

The invention involves inducing cancer cell division by administering to a mammal afflicted with cancer an IGF-1-receptor agonist, an insulin-receptor agonist, a growth hormone receptor agonist, and/or a sugar, at approximately the time radiation administered to enhance the effectiveness of anti-cancer radiation therapy. In the periods between radiation therapy, it is advisable to try to prevent cancer cell division. Thus, the invention can involve administering in the periods between radiation therapy an IGF-1 receptor antagonist (e.g., a monoclonal antibody against the IGF-1 receptor) or an insulin-receptor antagonist (e.g., a monoclonal antibody against the insulin receptor) to the mammal.

One embodiment involves administering a radiation-sensitizing agent described herein 0 to 12 hours before administering radiation to the mammal, and administering an IGF-1 receptor antagonist (or insulin-receptor antagonist) at a time outside of 0 to 12 hours before administration of radiation to the mammal.

One embodiment involves administering a radiation-sensitizing agent described herein between 6 hours before and 6 hours after administering radiation to the mammal, and administering an IGF-1 receptor antagonist (or insulin-receptor antagonist) at a time outside of 6 hours before to 6 hours after administration of radiation to the mammal.

One embodiment involves administering a radiation-sensitizing agent described herein between 3 hours before and 3 hours after administering radiation to the mammal, and administering an IGF-1 receptor antagonist (or insulin-receptor antagonist) at a time outside of 3 hours before to 3 hours after administration of radiation to the mammal.

The invention will now be illustrated by the following non-limiting examples.

### EXAMPLES

### Example 1

### In vitro Assays

MiaPaCa (a human pancreatic cancer cell line), LnCap (a human prostate cancer cell line), H226 and A549 (two human lung cancer cell lines) are grown in monolayer cultures in McCoy's 5A medium containing 10% fetal bovine serum and buffered with 2.0 g/L sodium bicarbonate and 0.02 M Hepes, pH 7.4. The cells are at seeded at 6×10⁴ cells per T30 flask. On day 3 the medium is changed. The cells are irradiated at either 4 days (exponential cells) or 7 days (plateau-phase cells). Twenty minutes before, 2 hours before, or immediately before irradiation, insulin (0.01, 0.1, or 1 μg/ml) or IGF-1 (3, 20, or 150 ng/ml) is added to the medium. Control cells have no added hormone. The cells are then irradiated with 200 rads X-rays. After irradiation, the cells are allowed to recover in the used medium for 60 minutes.

The cells are then typsinized and plated in fresh medium. The surviving fraction of a specific number of plated cells is determined by staining colonies with methylene blue after 7-10 days of growth.

These experiments show that at at least some concentrations and times before irradiation, both insulin and IGF increase cell killing by radiation in plateau-phase cells.

Exponential phase cells are also sensitized to radiation by both insulin and IGF-1.

Exponential-phase and plateau-phase cells are then treated with insulin and IGF-1 separately and together at their experimentally determined optimal concentrations immediately before, 15 minutes before, and 2 hours before irradiation with a range of radiation doses. Again, the cells are trypsinized and plated, and the percent surviving is determined. It is found that the effect of both hormones together is greater than either alone in enhancing killing.

Exponential- and plateau-phase cells are then treated with insulin at the optimal concentration in combination with 30 mM glucose. It is found that the addition of glucose enhances radiation killing above that observed with insulin alone.

### Example 2

### In vivo Assays

LnCap, MiaPaCa, H226, and A549 cells are grown in culture. Cells are harvested using 0.25% trypsin, washed, suspended in Dulbecco's PBS, and counted. 1×10⁶ cells are injected subcutaneously into the hind thigh of male nude mice. Tumors are grown to 250 mm³ in ˜21 days. Tumors are measured with a caliper, and the tumor size is calculated by the formula a²b/2, where a and b are the shorter and longer diameters of the tumor, respectively.

When the tumors reach 250 mm³, the mice are treated with insulin (5 or 40 μg/kg), IGF-1 (5 or 100 μg/kg) or insulin+glucose (5 g glucose/kg), or growth hormone (50 or 500 μg/kg), or saline control 30 minutes before radiation treatment.

The mice are anesthetized immediately before radiation treatment, and then the tumor-bearing leg is irradiated with X-rays at 1.4 Gy/minute, receiving a dose of 15 Gy.

The growth of tumor volume in the mice after irradiation is followed for 3 weeks.

These experiments will show insulin, insulin+glucose, IGF-1, and growth hormone all enhance the effectiveness of radiation therapy.

All patents, patent documents, and other references cited herein are incorporated by reference.

What is claimed is:

1. A method of treating cancer in a mammal comprising:
administering an IGF-1-receptor agonist to the mammal and administering radiation to the mammal, wherein the IGF-1-receptor agonist is not insulin.

2. The method of claim 1 wherein the IGF-1-receptor agonist has a greater affinity for the IGF-1 receptor than insulin.

3. The method of claim 1 wherein the IGF-1-receptor agonist is IGF-1.

4. The method of claim 1 wherein the IGF-1-receptor agonist is an IGF-1 variant that has reduced binding affinity for IGF-1 soluble binding proteins as compared to IGF-1.

5. The method of claim 4 wherein the IGF-1 variant is LONG-R3-IGF-1 (SEQ ID NO:1).

6. The method of claim 1 wherein the IGF-1-receptor agonist is administered before the radiation is administered.

7. The method of claim 6 wherein the IGF-1-receptor agonist is administered 0 to 6 hours before the radiation is administered.

8. The method of claim 7 wherein the IGF-1-receptor agonist is administered 15 minutes to 3 hours before the radiation is administered.

9. The method of claim 1 wherein the IGF-1 receptor agonist is administered between 3 hours before and 3 hours after the radiation is administered.

10. The method of claim 1 wherein the cancer is lung cancer (small cell or non-small cell), prostate cancer, colorectal cancer, breast cancer, pancreatic cancer, leukemia, liver cancer, stomach cancer, ovarian cancer, uterine cancer, testicular cancer, brain cancer, non-Hodgkin's lymphoma, Hodgkin's lymphoma, Ewing's sarcoma, osteosarcoma, neuroblastoma, rhabdomyosarcoma, melanoma, head or neck cancer, or brain cancer.

11. The method of claim 9 wherein the cancer is low-grade non-Hodgkin's lymphoma.

12. The method of claim 1 wherein the mammal is a human.

13. A method of treating cancer in a mammal comprising:
administering a growth hormone-receptor agonist to the mammal and administering radiation to the mammal.

14. The method of claim 13 wherein the growth hormone-receptor agonist is growth hormone.

15. The method of claim 14 wherein the growth hormone-receptor agonist is human growth hormone.

16. The method of claim 13 wherein the mammal is a human.

17. The method of claim 13 wherein the growth hormone-receptor agonist is administered zero to six hours before the radiation is administered.

18. The method of claim 17 wherein the growth hormone-receptor agonist is administered 15 minutes to 3 hours before the radiation is administered.

19. The method of claim 13 wherein the cancer is lung cancer (small cell or non-small cell), prostate cancer, colorectal cancer, breast cancer, pancreatic cancer, leukemia, liver cancer, stomach cancer, ovarian cancer, uterine cancer, testicular cancer, brain cancer, non-Hodgkin's lymphoma, Hodgkin's lymphoma, Ewing's sarcoma, osteosarcoma, neuroblastoma, rhabdomyosarcoma, melanoma, head or neck cancer, or brain cancer.

20. The method of claim 19 wherein the cancer is low-grade non-Hodgkin's lymphoma.


SEQUENCE LISTING (1 sequence)

SEQ ID NO: 1 - protein, 82 aa, artificial
  LONG-R3-IGF-1 synthetic variant of IGF-1
MFPAMPLSSLFVNGPRTLCGAELVDALQFVCGDRGFYFNKPTGYSSSRRAPQTGIVDECCFRSCDLRRLEMYCAPLKPAKSA